(19) United States
(12) Patent Application Publication
(10) Pub. No.: US 2024/0150854 A1
(43) Pub. Date: May 9, 2024

(54) METHODS AND SYSTEMS FOR DETECTION OF MONKEYPOX VIRUS

(71) Applicant: Laboratory Corporation of America Holdings, Bulington, NC (US)

(72) Inventors: Thomas Jacob Urban, Mebane, NC (US); Ayla Burns Harris, Stokesdale, NC (US); Jonathan David Williams, Hillsborough, NC (US); Deborah John Boles, Cary, NC (US); Timothy Staton, Burlington, NC (US); Nathan Harris, Mebane, NC (US); Phyllis Davis, Burlington, NC (US); Suzanne Dale, Burlington, NC (US)

(21) Appl. No.: 18/500,048

(22) Filed: Nov. 1, 2023

Related U.S. Application Data
(60) Provisional application No. 63/421,345, filed on Nov. 1, 2022.
(60) Provisional application No. 63/491,652, filed on Mar. 22, 2023.
(60) Provisional application No. 63/591,611, filed on Oct. 19, 2023.

(51) Int. Cl. C12Q 1/70; C12Q 1/6806

(52) U.S. Cl. CPC C12Q 1/701; C12Q 1/6806; C12Q 2600/16

(57) ABSTRACT

Disclosed are methods and systems to detect a class of non-variola Orthopoxviruses, and, more particularly, monkeypox virus. In certain embodiments, the method may comprise obtaining a sample from the subject; and detecting a nucleic acid sequence specific to the non-variola Orthopoxvirus. In some embodiments, the method may further isolation of the non-variola Orthopoxvirus from a solid substrate such as a tissue swab and further purification of viral nucleic acid using silica beads. The sample may in some cases be self-collected by a subject. The disclosed methods and systems, including kits for self-collection, may be used for a high throughput assay allowing for screening of hundreds of samples per day.

### CROSS-REFERENCE TO RELATED APPLICATIONS

This application claims priority to U.S. Provisional Application No. 63/421,345 filed Nov. 1, 2022, U.S. Provisional Patent Application No. 63/491,652 filed Mar. 22, 2023, and U.S. Provisional Patent Application No. 63/591,611 filed Oct. 19, 2023, each of which are incorporated by reference herein in their entireties.

### REFERENCE TO A SEQUENCE LISTING

A Sequence Listing conforming to the rules of WIPO Standard ST.26 is hereby incorporated by reference. Said Sequence Listing has been filed as an electronic document via Patent Center encoded in XML in UTF-8 text. The electronic document, created on Oct. 26, 2023, is entitled “LC 2022-14-US-1 (057618-1411998).xml”, and is 6,463 bytes in size.

### TECHNICAL FIELD

The present disclosure relates generally to methods and systems for detection of viruses. In certain embodiments, the virus may be from a class of Orthopoxviruses, and more particularly, a monkeypox virus.

### BACKGROUND

Monkeypox is an infectious disease that can occur in humans and some animals. Symptoms include fever, swollen lymph nodes and a rash that can cause blisters. Monkeypox, like Cowpox, is a non-variola virus in the genus of Orthopoxvirus.

Variola virus, also a member of the Orthopoxvirus genus, is the causative agent of smallpox and was certified eradicated in 1980 by the World Health Organization. Smallpox vaccinations were ceased world-wide as a result. However, in recent years, concerns over the potential use of Variola virus as a biological weapon led the United States to resume smallpox vaccinations on a limited basis. As the ACAM2000 smallpox vaccine contains live Vaccinia virus, it is possible for vaccine recipients and/or their close contacts to develop adverse reactions to the vaccine including the emergence of pustules on the skin. Vaccinia infection is not known to occur naturally in the United States. Rather it occurs in conjunction with smallpox vaccination or contact with a smallpox vaccine recipient.

Monkeypox had been limited to locations outside the United States, with the exception of the 2003 monkeypox outbreak associated with prairie dogs. However, in May 2022, multiple cases of monkeypox were reported in several countries that do not normally report Monkeypox, including the United States. Monkeypox is the only circulating non-variola Orthopoxvirus in mammalian systems. At this time, there is no known cure, although two vaccines (JYNNEOS and ACAM2000) are available.

In August of 2022 the number of monkeypox cases was at about 400 new cases per day. As such, methods for high-throughput screening and that can detect monkeypox, or other non-variola Orthopoxvirus, as part of the pathogenic Orthopoxviruses is needed.

### BRIEF SUMMARY

Described herein are methods and systems to accurately determine the presence of a particular virus in a biological sample in a high throughput manner. In certain embodiments, the virus is a non-variola Orthopoxvirus. In certain embodiments, the virus is monkeypox. The methods and systems may be used to detect commonly known human pathogenic Orthopoxviruses without detecting the Variola virus, the causative agent of smallpox. The methods and systems may be embodied in a variety of ways.

In certain embodiments, disclosed is a method for detecting the presence of a virus, such as a non-variola Orthopoxvirus in a subject comprising: obtaining a sample from the subject; and detecting a nucleic acid sequence specific to the virus (e.g., a non-variola Orthopoxvirus). In certain embodiments, the method may comprise the step of detecting a second nucleic acid sequence that serves as a control. The methods may be used as part of a high-throughput screening program.

In certain embodiments, disclosed is a system for detecting the presence of a virus, such as a non-variola Orthopoxvirus in a subject comprising:
(a) a component for isolation of the non-variola Orthopoxvirus from a solid substrate comprising a sample from the subject using a multi-well procedure;
(b) a component for purification of a nucleic acid sequence specific to the non-variola Orthopoxvirus;
(c) a component for PCR amplification of the nucleic acid sequence specific to the non-variola Orthopoxvirus; and
(d) a component to output results indicating a presence or absence of the nucleic acid sequence specific to the non-variola Orthopoxvirus.

Also disclosed is a non-transitory, computer medium comprising instructions to perform any of the steps of the disclosed methods or to run any of the components of the disclosed systems.

Features described with respect with one embodiment can be incorporated with other embodiments although not specifically discussed therewith. As such, aspects of the disclosure described with respect to one embodiment, may be incorporated in a different embodiment although not specifically described relative thereto. That is, all embodiments and/or features of any embodiment can be combined in any way and/or combination. The foregoing and other aspects of the present disclosure are explained in detail in the specification set forth below.

### BRIEF DESCRIPTION OF THE DRAWINGS

The present disclosure may be better understood with reference to the accompanying non-limiting drawings. The order of operations and/or steps illustrated in the figures or recited in the claims are not intended to be limited to the order presented unless stated otherwise.

FIG. 1 shows a method for determining the presence of a non-variola Orthopoxvirus in a samples obtained from an individual or individual subjects in accordance with an embodiment of the disclosure.

FIG. 2 shows a system for determining the presence of a non-variola Orthopoxvirus in samples obtained from an individual or individual subjects in accordance with an embodiment of the disclosure.

FIG. 3 shows instructions for self-collecting a sample in accordance with an embodiment of the disclosure.

### DETAILED DESCRIPTION

### Terms and Definitions

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer, or section. Thus, a first element, component, region, layer, or section discussed below could be termed a second element, component, region, layer or section without departing from the teachings of the disclosure. The sequence of operations (or steps) is not limited to the order presented in the claims or figures unless specifically indicated otherwise.

The term “programmatically” means carried out using a computer program and/or software, processor or ASIC directed operations. The term “electronic” and derivatives thereof refer to automated or semi-automated operations carried out using devices with electrical circuits and/or modules rather than via mental steps and typically refers to operations that are carried out programmatically.

The terms “automated” and “automatic” means that the operations can be carried out with minimal or no manual labor or input. The term “semi-automated” refers to allowing operators some input or activation, but calculations, acquisition, purification, and other steps are done electronically, typically programmatically, without requiring manual input.

The term “about” refers to +/−10% (mean or average) of a specified value or number.

The term “protocol” refers to an automated electronic algorithm (typically a computer program) with mathematical computations, defined rules for data interrogation and analysis that manipulates a system to perform a set of instructions.

As used herein, “CT” or “ct” refers to cycle threshold, or the total number of cycles required to amplify and detect a nucleic acid target (e.g., a viral nucleic acid) by real time-PCR and/or PCR.

As used herein, repeatability (or intra-assay precision) describes the closeness of agreement between results of successive measurements of the same analyte and carried out under the same conditions of measurement. Intra-assay repeatability is the measurement of the variability when the same specimen is analyzed during one analytical run.

As used herein reproducibility (or inter-assay precision) describes the closeness of agreement between results of successive measurements of the same analyte and carried out under the same conditions of measurement. Inter-assay repeatability is a measurement of the variability when the same specimen is analyzed during more than one run.

As used herein, real-time PCR or quantitative PCR (qPCR) allows for real-time detection of a PCR amplification product. Real-time polymerase chain reaction (PCR) assays use a fluorescent-labeled probe or intercalating dye to visualize a PCR reaction and monitor the quantity of double-stranded DNA product that is produced. The fluorogenic 5′ nuclease assay (i.e., TaqMan® assay) is a real-time PCR assay which uses a fluorogenic probe, consisting of an oligonucleotide with a reporter dye attached to the 5′ end and a quencher dye attached at or near the 3′ end. The probe anneals to a specific target sequence located between the forward and reverse primers. During the extension phase of the PCR cycle, the 5′ nuclease activity of Taq polymerase degrades the probe, causing the reporter dye to separate from the quencher dye and a fluorescent signal is generated. With each cycle, additional reporter dye molecules are cleaved from their respective probes, and the fluorescence intensity is monitored during the PCR. The Taq polymerase used may be inactive at room temperature and activated by incubation at 95° prior to initiating the cycling portion of the assay. This minimizes the production of nonspecific amplification products.

“Swab” and “dry swab” as used refers to a physical vector for containing and/or obtaining a sample from an individual. The vector may be constructed of various materials and may encompass cotton swab balls, tissues, plastic forceps, plastic inoculating loops, popsicle sticks, dry or moist swab sticks, and others of the like.

“Sample” or “patient sample” or “biological sample” or “specimen” are used interchangeably herein. Non-limiting examples of samples that may be used for analysis with the disclosed methods and systems include, blood or a blood product (e.g., serum, plasma, or the like), urine, nasal swabs, a liquid biopsy sample, skin swabs, lesion swabs, or combinations thereof. In some cases, DNA may be extracted from lesion material, such as lesion fluid on a dray swab, lesion fluid swab in viral transport media, lesion fluid on a slide, crust or lesion roof. The term “blood” encompasses whole blood, blood product or any fraction of blood, such as serum, plasma, buffy coat, or the like as conventionally defined. Suitable samples include those which are capable of being deposited onto a substrate for collection and drying including, but not limited to: blood, plasma, serum, urine, saliva, tear, cerebrospinal fluid, organ, hair, muscle, puss, or other tissue samples or other liquid aspirates. The term “biological sample” further refers to a sample obtained from a biological source, including, but not limited to, an animal, a cell culture, an organ culture, tissue, and the like.

The terms “at home collection” or “self-collection” and the like refer to the use of a component or components that may be provided to a subject for self-collecting a sample. In some cases, self-collection may be performed with a kit containing a swab, a container to hold the swab after a sample has been taken and optionally, having a transport fluid (e.g., buffer), a biohazard packaging container, and optionally a container to return the self-collected sample to a laboratory for testing.

The term “individual”, “patient”, or “subject” is used broadly and refers to an individual that provides a sample for testing or analysis. The “individual” or “patient” or “subject” from whom a sample is collected, obtained, and/or provided by, includes any and all warm-blooded mammalian subjects such as humans and/or animals.

### Methods for Detecting Non-Variola Orthopoxvirus

The present disclosure will now be described more fully hereinafter, in which embodiments of the invention are shown. This disclosure may, however, be embodied in different forms and should not be construed as limited to the embodiments set forth herein. Rather, these embodiments are provided so that this disclosure will be thorough and complete, and will fully convey the scope of the invention to those skilled in the art.

Described herein are methods for detecting the presence of a virus, such as a non-variola Orthopoxvirus in an individual. In certain embodiments, the non-variola Orthopoxvirus is monkeypox (Mpox). Steps for detecting the presence of a non-variola Orthopoxvirus in an individual may comprise of: (a) obtaining a sample from the subject and (b) detecting a nucleic acid sequence specific to a non-variola Orthopoxvirus, and optionally (c) detecting a second nucleic acid sequence that serves as a control. In an embodiment, the nucleic acid sequence is a sequence that is not specific for monkeypox but may be used as a monkeypox diagnostic due to the lack of other circulating pox viruses (e.g., camelpox, cowpox). In certain embodiments, the sample may be self-collected by the individual, e.g., at his or her home, and mailed back to a laboratory for testing.

In some embodiments a method for detecting a presence of a non-variola Orthopoxvirus in a subject may include the steps of obtaining a sample from the subject and determining whether a nucleic acid sequence specific to a non-variola Orthopoxvirus is present in the DNA of the sample. Various methods for determining the presence of a nucleic acid or a nucleic acid sequence may be applied. Some embodiments may include a real-time polymerase chain reaction (i.e., quantitative or q-PCR) step to amplify the DNA extracted from the sample. Real-time PCR may be performed on samples individually or using multiplex PCR. Or other methods such as amplification by PCR followed by DNA sequencing may be used.

Thus, in certain embodiments, each extracted DNA sample is analyzed with each primer/probe set separately. Or PCR may be performed using a multiplex PCR reaction allowing for amplification of the non-variola Orthopoxvirus (e.g., VAC primers and probes) as well as controls (e.g., RNaseP (RP) primers and probes and/or vaccinia primers and probes). For multiplex real time PCR different fluorophores may be used to detect different amplification products. Or, in certain embodiments, the same fluorophore may be used.

In certain embodiments, each extracted DNA sample is analyzed with each primer/probe set separately. DNA samples may be tested using both a target (VAC1) primer and probe set and a control primer and probe set (RP). The VAC1 primers and probe may target the essential poxvirus gene, “E9L.” Since E9L is an essential gene, changes within the targeted sequence are less likely to occur. Therefore, the methods are not likely to yield a false negative by virtue of a genetic change within the virus. In certain embodiments, the disclosed method does not detect Variola virus because the probe has 3 base pair mismatches to the Variola virus sequence corresponding to the probe. The RP primer/probe set targets the human RNase P gene. Human DNA should be present in DNA extracted from clinical specimens containing cellular material. The amplification of RNase P thus serves as a control to ensure adequate DNA results from the extraction of clinical specimens, and/or to ensure proper function of common reagents and equipment, and/or to demonstrate the absence of inhibitory substances.

In certain embodiments, primers and probes approved by and available from the Center for Disease Control (CDC) are used. Thus, in certain embodiments, the method may use the Non-variola Orthopoxvirus Real-Time PCR Primer and Probe Set (VAC1) primers and probes (CDC KT0035A) and the RNase P Real-Time PCR Primer and Probe Set (RP) CDC KT0068. Also in certain embodiments, a positive control is used. For example, in certain embodiments the vaccinia virus (e.g., Vaccinia (MVA)) (VAC-1 positive control) from ATCC (VR-1508) may be used, as vaccinia virus may represent a hybrid of variola and cowpox). Alternatively, BEI or ATCC extracted genomic (e.g., Vaccinia (MVA) Genomic, ATCC; catalog #VR-1508D) may be used. Also in certain embodiments, a RNase P positive control (i.e., PCR amplification of the RNase-P gene) is used to ensure adequate DNA yield with specimen collection or the DNA extraction for clinical samples. Or other human DNA controls (e.g., β-actin, Glyceraldehyde-3-phosphate dehydrogenase (GAPDH) may be used. Also, in certain embodiments, an extraction control (EC) may be used as a control for the extraction process and should be treated as a clinical specimen throughout the extraction and PCR process.

In certain embodiments, the VAC1 primers and probe target the poxvirus gene, E9L. Since E9L is considered an essential gene to the virus, changes within the targeted sequence are less likely to occur. For example, an RNase P Real-Time PCR Primer and Probe Set (RP) CDC KT0068 may be used. In certain embodiments the non-variola orthopoxvirus forward primer may be 5′-TCAACTGAAAAGGCCATCTATGA-3′ (SEQ. ID NO. 1), the reverse primer may be 5′-GAGTATAGAGCACTATTTCTAAATCCCA-3′ (SEQ. ID NO. 2), and the probe sequence may be 5′-FAM-CCATGCAATA(T-BHQ1)ACGTACAAGATAGTAGCCAAC-Phos-3′ (SEQ ID NO. 3). The human DNA RNase P control forward primer may be 5′-AGATTTGGACCTGCGAGCG-3′ (SEQ. ID NO. 4), the RNase P reverse primer may be 5′-GAGCGGCTGTCTCCACAAGT-3′ (SEQ. ID NO. 5), and the RNase P probe may be 5′-FAM-TTCTGACCTGAAGGCTCTGCGCG-BHQ1-3′ (SEQ. ID NO. 6). Real-time PCR may be performed using optimized cycling procedures depending on the primers and probes used. For example, in one embodiment, the PCR may comprise an initial denaturation step at 95° C. for 20 seconds (i.e., 1 cycle) followed by 40 cycles of 95° C. for 3 seconds and 63° C. for 30 seconds. See e.g., Jun. 6, 2022: Lab Advisory: CDC Publishes Non-variola Orthopoxvirus PCR Testing Procedure. For example, in another embodiment, the PCR may comprise an initial denaturation step at 95° C. (i.e., 1 cycle) followed by 40 cycles of 95° C. for 5 seconds, and 63° C. for 30 seconds.

Due to the primer/probe design and amplification conditions, the assay is not likely to yield a false negative by virtue of a genetic change within the virus. In certain embodiments, the assay does not detect Variola virus because the probe has 3 base pair mismatches within the corresponding Variola virus sequence. The RP primer/probe set targets the human RNase P gene. Human DNA present in DNA extracted from clinical specimens containing cellular material may be used to determine the overall quality of the sample and assay. Thus, in an embodiment, the human RNase P assay thus serves as a control to ensure adequate DNA yield from the extraction of clinical specimens, as well as to ensure proper function of common reagents and equipment, and to demonstrate the absence of inhibitory substances.

Embodiments of the present disclosure may include samples obtained from a human or an animal subject. In certain embodiments, the step of detecting a nucleic acid sequence specific to the non-variola Orthopoxvirus (e.g., a monkeypox virus) comprises isolation of virion particles from a solid substrate. In one embodiment, the sample may be obtained from a dry swab of skin or a skin lesion site. The sample may be obtained by vigorously swabbing the skin and/or skin lesion such that virus particles are present in large quantities on the swab. In certain embodiments, the sample may be self-collected by the individual, e.g., at his or her home, and mailed back to a laboratory for testing.

Thus, in some embodiments, the sample comprises virions (e.g., Monkeypox virus) present on a swab (e.g., a tissue swab) from a skin lesion. Such samples may comprise free virus as well as cells infected with virus. Swab samples may then be placed in a collection tube or other receptacle (e.g., bag) and stored at 2-8° C. or at ambient temperature. Samples self-collected by an individual may be mailed to a central laboratory for testing.

In certain embodiments, isolation of the virus particles (i.e., monkeypox virions) from such solid substrates (e.g., swabs) comprises a multi-well extraction procedure. In certain embodiments, a Slicprep™ 96 Device (Promega, Madison, WI) may be used for extracting virus from a plurality of tissue swabs each obtained from different individuals or lesions (e.g., multiple lesions from the same subject). Thus, in an embodiment, individual swabs are placed in individual wells of the device to allow for isolation of virions from the plurality of individual swabs. Or, samples may be processed individually (i.e., one at a time) to isolate virions from the sample.

In certain embodiments, the method includes the step of extracting nucleic acid from the isolated virions. Thus, in certain embodiments, extraction of the nucleic acid from virions isolated from the solid substrate may comprise lysis of the virions followed by binding of the nucleic acid present in the sample to silica beads. In one embodiment, a MagNA Pure 96 well-plate (MP96, Roche Diagnostics, Indianapolis, IN) is used for isolation of nucleic acids from a sample for further study. Thus, using MagNA Pure (Roche Diagnostics, Indianapolis, IN), the sample material is lysed, nucleic acids are released, and nucleases are denatured. Next the sample may be incubated with silica beads such that the nucleic acids bind to the silica surface of magnetic glass particles (MGPs) in the presence of isopropanol and high conditions of chaotropic salts, which remove water from hydrated molecules in solution. At this point, MGPs with bound nucleic acids may be magnetically separated from the residual lysed sample and unbound substances, such as polysaccharides, proteins, cell debris, and PCR inhibitors removed by subsequent washing. Purified nucleic acids may be eluted from the MGPs by applying low-salt conditions and heat.

Embodiments of the methods allow for high throughput screening for a non-variola Orthopoxvirus (e.g., a monkeypox virus). Using the disclosed multiwell extraction process for virion isolation, followed by multiwell purification of viral nucleic acid and/or multiplex PCR allows for assaying multiple samples individually. For example, in certain embodiments, 93 individual samples may be processed in a single 96-well plate. This can allow for a single laboratory to process hundreds (e.g., 4 or more plates) per day. For example, in certain embodiments, the methods allow for processing at least 20, or 40, or 60, or 80, or 100, or 150, or 200, or 250, or 300, or 350, or 400, or 450, or 500 or more samples in one day. Additionally and/or alternatively, in some cases duplicates or triplicates of a sample may be evaluated.

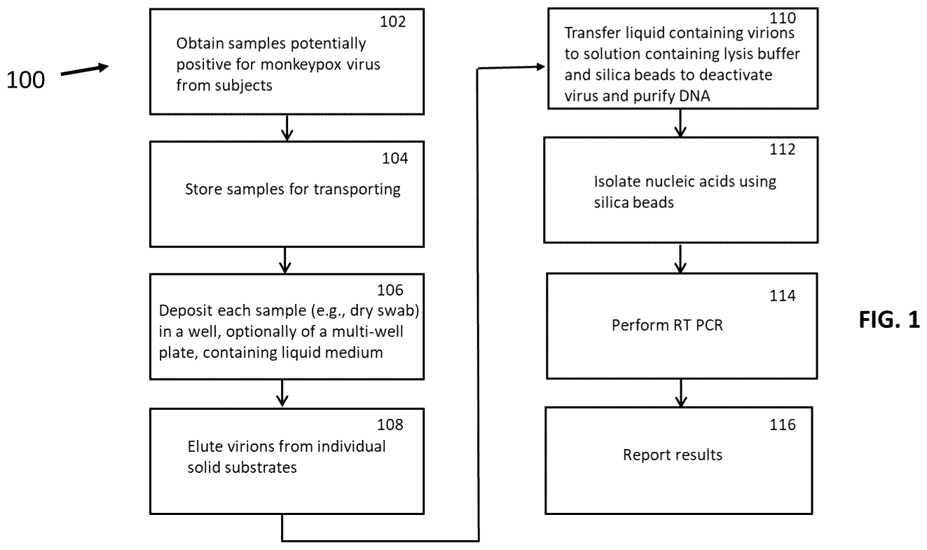

FIG. 1 shows an embodiment of the disclosed method 100. As shown in FIG. 1, a specimen or specimens used for sample collection may be a moistened or a dry swab obtained from an individual 102. Swabs may be synthetic tip swabs with aluminum or plastic shafts. Swabs may be used with or without media. In certain embodiments, two specimens may be obtained such that one can be stored while the other can be studied. In one embodiment, the dry swab containing the sample may be placed in a container for storing, handling, and transporting 104. A specimen obtained for future study can be stored in cold temperatures at 2-8° C. or −20° C. In one embodiment, the swab may be obtained and placed in liquid viral media. Liquid viral media for containing samples may be universal transport media (UTM®) or viral transport media (VTM). As disclosed herein, in some cases, the sample may be self-collected by the subject (i.e., individual) wishing to determine if he or she may have an active viral infection. Such self-collected samples may be mailed to a laboratory for analysis.

In one embodiment, the dry swab containing the sample may be placed in a container for storing, handling, and transporting 104 as disclosed herein. Upon receipt, the dry swab containing the sample may be deposited and submerged in a well of a multi-well plate containing liquid medium 106. In some examples, the multi-well plate may be a Promega Slicprep™ plate (Promega, Madison, WI). The liquid medium in the well plate may be a phosphate buffered solution (PBS) or a saline solution or the like. In one embodiment, the plate may be a 96 well plate where one or more patient swabs (e.g., 96) may be analyzed concurrently. Further, both experimental and control wells may be contained within the same well plate for increased inter-assay and control consistency. The dry swab deposited in the buffer may be eluted for about 10 minutes at ambient temperature 108. In one embodiment, following a 10-minute elution time, the multi-well plate may be subject to centrifugation at 1900×g for one minute to ensure sample elution is collected at the multi-well plate bottom.

In certain embodiments, following elution of the specimen contents from the swab, 100 μL of the extracted contents from each well of the Promega Slicprep™ (Promega, Madison, WI) plate may be transferred into individual wells of a MP96 well-plate (Roche Diagnostics, Indianapolis, IN), each well containing about 100 μL of a lysis buffer 110. Proper homogenization may be ensured by pipetting gently. The sample mixed with lysis buffer may be incubated at room temperature for about 15 minutes to deactivate the virus. In some embodiments, a further step of centrifuging may follow such that the deactivated virus may be concentrated at the bottom of the container or multi-well plate. Centrifuging of deactivated virus may be at 500×g for 30 seconds in some examples. Or, similar conditions may be used.

In another embodiment, the isolation of viral nucleic acid may be performed in a tube such as a conical tube. In one example, the conical tube may be a part of a swab extraction tube system (SETS) tube protocol. For example, the dry swab may be deposited in a tube with a volume of PBS and allowed to elute for about 10 minutes. The sample-eluted PBS volume may be transferred to a second tube via aerosol-barrier micropipette tips to minimize cross contamination. The tube may then be centrifuged at 1900×g for one minute to ensure sample elution is collected at the conical tube bottom. This embodiment may be preferred for a smaller set of samples that do not require a multi-well plate for virion isolation.

Next, about 100 μL of the extracted contents from the SETS tube may be transferred into a MP 96 well-plate containing 100 μL of a lysis buffer. Proper homogenization may be ensured by pipetting gently. The sample mixed with lysis buffer may be incubated at room temperature for about 15 minutes or greater to deactivate the virus. In some embodiments, a further step of centrifuging may follow such that the deactivated virus may be concentrated at the bottom of the container or multi-well plate. Centrifuging of deactivated virus may be spun at 500×g for 30 seconds in some examples.

In an alternative embodiment, the substrate containing the sample of the subject may be submitted in liquid viral media in a container (UTM/VTM). A sample submerged in liquid viral media can be vortexed to ensure proper homogenization of sample. The sample submerged in liquid viral media may be centrifuged at 1900×g for one minute to ensure liquid sample is collected at the bottom of the conical container.

In an embodiment, following the transfer of viral contents into the MP96 plate containing the lysis buffer 110, and following incubation and centrifugation, isolation of nucleic acids using silica beads may follow 112. Nucleic acids may bind to a silica surface of the MGPs, which allows for unbound substances, such as cell debris, to be removed in subsequent washes. Nucleic acids may then be eluted from the MGPs by using heat and low-salt conditions. In some embodiments, after sample DNA extraction and isolation is performed, a step of performing PCR may follow 114. In certain embodiments, real-time PCR (i.e., quantitative PCR or qPCR) is used. In certain embodiments, the qPCR assays use a fluorescent-labeled probe or intercalating dye to visualize a PCR reaction and monitor the quantity of double stranded DNA product that is produced.

In certain embodiments, the qPCR uses a fluorogenic probe, consisting of an oligonucleotide with a reporter dye attached to the 5′ end and a quencher dye attached at or near the 3′ end. The probe anneals to a specific target sequence located between the forward and reverse primers. During the extension phase of the PCR cycle, the 5′ nuclease activity of Taq polymerase degrades the probe, causing the reporter dye to separate from the quencher dye and a fluorescent signal is generated. With each cycle, additional reporter dye molecules are cleaved from their respective probes, and the fluorescence intensity is monitored during the PCR. In an embodiment, the Taq polymerase is inactive at room temperature and is activated by incubation at 95° C. prior to initiating the cycling portion of the assay. This minimizes the production of nonspecific amplification products and undesired Taq enzymatic activity during the amplification setup. A variety of fluorophores and/or quencher dyes may be employed for real time PCR. In certain embodiments, a detectable moiety is a fluorescent dye. A fluorescent detectable moiety can be stimulated by a laser with the emitted light captured by a detector. The detector can be a charge-coupled device (CCD) or a confocal microscope, which records its intensity.

Suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM), hexachloro-fluorescein (HEX), carbocyanine, merocyanine, styryl dyes, oxonol dyes, phycoerythrin, erythrosin, eosin, rhodamine dyes (e.g., carboxytetramethylrhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR)), coumarin and coumarin dyes (e.g., methoxycoumarin, dialkylaminocoumarin, hydroxycoumarin, aminomethylcoumarin (AMCA)), Q-DOTS, Oregon Green Dyes (e.g., Oregon Green 488, Oregon Green 500, Oregon Green 514), Texas Red, Texas Red-X, SPECTRUM RED, SPECTRUM GREEN, cyanine dyes (e.g., CY-3, CY-5, CY-3.5, CY5.5), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), IRDyes (e.g., IRD40, IRD 700, IRD 800), and the like. Favorable properties of fluorescent labeling agents include high molar absorption coefficient, high fluorescence quantum yield, and photostability. In some embodiments, labeling fluorophores exhibit absorption and emission wavelengths in the visible (i.e., between 400 and 750 nm) rather than in the ultraviolet range of the spectrum (i.e., lower than 400 nm).

A detectable moiety may include more than one chemical entity such as in fluorescent resonance energy transfer (FRET). Resonance transfer results an overall enhancement of the emission intensity. To achieve resonance energy transfer, the first fluorescent molecule (the “donor” fluor) absorbs light and transfers it through the resonance of excited electrons to the second fluorescent molecule (the “acceptor” fluor). In one approach, both the donor and acceptor dyes can be linked together and attached to the oligo primer. Donor/acceptor pairs of dyes that can be used include, for example, fluorescein/tetramethylrohdamine, IAEDANS/fluorescein, EDANS/DABCYL, fluorescein/fluorescein, BODIPY FL/BODIPY FL, and Fluorescein/QSY 7 dye. Many of these dyes also are commercially available, for instance, from Molecular Probes Inc. (Eugene, Oreg.). Suitable donor fluorophores include 6-carboxyfluorescein (FAM), tetrachloro-6-carboxyfluorescein (TET), 2′-chloro-7′-phenyl-1,4-dichloro-6-carboxyfluorescein (VIC), and the like.

Or, suitable fluorescent quencher molecules may be used. As used herein, fluorescent quenching refers to any process that decreases the fluorescence of a molecule such as black hole quenchers commercially available from Biosearch Technologies. Such quenchers include, but are not limited to, BHQ0, BHQ1, BHQ3, and BHQ4. Different quencher dyes are suitable for use with specific fluorophores, including FAM, TET, JOE, HEX, Oregon Green®, TAMRA, ROX, Cyanine-3, Cyanine-3.5, Cyanine-5 and Cyanine-5.5 (e.g., CY-3, CY-5, CY-3.5, CY5.5).

In some embodiments, a positive control comprising extracted vaccinia DNA with a CT range of 30-36 may be used as a comparison. The positive control may be referred to as the positive amplification control (PAC). In some embodiments, PCR-grade water may serve as the negative control for the assay in the no template control (NTC). In some embodiments, a second negative control with a non-monkey pox template may be included. Such a control may be referred to as a negative extraction control (NEC) and may comprise a separate control primer and probe set controlling for specimen quality. The NEC may target a human RNase P gene and thus may be referred to as the RP control.

In one embodiment, all experimental and control samples may be positioned in a multi-well plate such as a 96 well-plate. Plate may be subject to centrifugation by a plate centrifuge between adding and mixing of samples. Samples, once prepared, may be optimized to perform PCR manually or automatically through various types of instrumentations. The results may then be interpreted and reported to the individual and/or his or her medical care provider 116.

### Systems for Detecting Non-Variola Orthopoxvirus

Also disclosed herein are systems for performing any of the steps of the disclosed methods and computer-implemented instructions for performing any of the steps of the disclosed methods or running any of the parts of the disclosed systems.

For example, disclosed is a system comprising one or more stations and/or components for performing any of the previous method embodiments comprising a station or a component for performing a step or steps of the method. In certain embodiments any one of the stations or components may be controlled by a computer. In certain embodiments, disclosed is a system comprising a component or components configured to perform at least some of the steps of: (a) isolating a nucleic acid sequence specific to a non-variola Orthopoxvirus from a sample, that is optionally a solid substrate; (b) purifying a specific nucleic acid sequence of the non-variola Orthopoxvirus, and (c) performing PCR amplification of a nucleic acid sequence specific to the non-variola Orthopoxvirus. In certain embodiments, the non-variola Orthopoxvirus is monkeypox. Thus, in certain embodiments, disclosed is a system for detecting the presence of a non-variola Orthopoxvirus in a subject comprising: (a) a component for isolation of the non-variola Orthopoxvirus from a solid substrate using a multi-well procedure; (b) a component for purification of a nucleic acid specific to the non-variola Orthopoxvirus; (c) a component for PCR amplification of the nucleic acid sequence specific to the non-variola Orthopoxvirus; and (d) a component to output results indicating a presence or absence of the nucleic acid sequence specific to the non-variola Orthopoxvirus. Or, the system may be used for the detection of other viruses in a high throughput manner, particularly where samples are obtained using a solid substrate.

In certain embodiments, the system is, or may include, a kit for self-collection of viral samples. The kit may comprise a swab, or a plurality of swabs for collection of samples from a lesion or lesions on a subject's skin. The kit may further comprise a transport container (i.e., transport tube) comprising a fluid (e.g., buffer). The transport container may allow for submersion of the swab into the container and a cap for secure sealing of the container. The kit may further comprise packaging (e.g., biohazard bag, external packaging, and mailing instructions) for the subject to mail his or her specimen to a laboratory for testing. The kit may further comprise instructions for use. Once at the laboratory, the swab may be removed from the container and processed using the methods and systems disclosed herein. In certain embodiments, the kit may comprise a shipping box, pre-labeled return envelope, collection and shipping directions, specimen collection materials (swab and transport tube), a specimen biohazard bag containing an absorbent sheet, and a specimen confirmation form. Instructions may be included in the kit to direct the home users on how to appropriately collect the lesion swab specimen and place it in the transport tube, how to properly package the specimen and how to mail the specimen back to the laboratory using a pre-labeled return envelope (e.g., FedEx or other carriers).

In one embodiment, the forgoing method steps may be performed by a system configured to perform the method steps sequentially in one system. Alternatively, the method steps may be performed sequentially in more than one system with any method performed by any of the one or more systems.

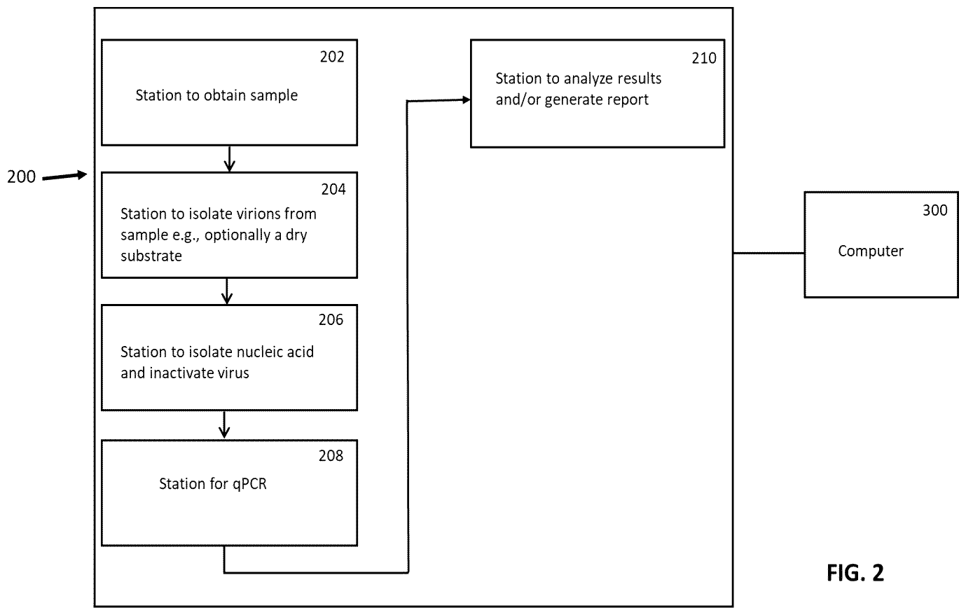

FIG. 2 shows an illustration of a system 200 of the disclosure. In one embodiment, the system 200 may comprise a component (or station) to obtain a sample 202. As disclosed herein, the sample may comprise a dry swab obtained from a lesion of the skin from a subject. Thus, in some cases the sample comprises virions (e.g., monkeypox virus) present on a swab (e.g., a tissue swab) from a skin lesion. Swab samples may then be placed in a collection tube or other receptacle (e.g., bag) and stored at 2-8° C. or ambient temperature. Or the sample may comprise a swab that has been self-collected by the subject and mailed to the laboratory using a transport container of the disclosed kits.

The system may also include a station to extract the virus from the dry swab 204. In certain embodiments, isolation of the virus particles (i.e., monkeypox virions) from a solid substrate (e.g., a swab) comprises a component for multi-well extraction. For example, in certain embodiments, up to 96 samples can be extracted using a single multiwell extraction component. In certain embodiments, a Slicprep 96 Device (Promega, Madison, WI) may be used for extracting virus from a plurality of tissue swabs each obtained from different individuals or lesions (e.g., multiple lesions from the same subject). Thus, in an embodiment, individual swabs are placed in individual wells of the device to allow for isolation of virions from the plurality of individual swabs. Or, samples may be processed individually (i.e., one at a time) to isolate virions from the sample.

Additionally and/or alternatively, the system may comprise a component or a station to extract nucleic acid from the virus obtained from the sample 206. Thus, in certain embodiments, extraction of the nucleic acid from virions isolated from the solid substrate may comprise lysis of the virions followed by binding of the nucleic acid present in the sample to silica beads. In one embodiment, a MagNA Pure 96 well-plate (MP96, Roche Diagnostics, Indianapolis, IN) is used for isolation of nucleic acids from a crude sample for further study. Thus, using MagNA Pure (Roche Diagnostics, Indianapolis, IN), aliquots of virions isolated from individual samples material are lysed, nucleic acids are released, and nucleases are denatured. Next the sample is incubated with silica beads (e.g., magnetic glass particles or MGPs) such that the nucleic acids bind to the silica surface of the MGPs in the presence of isopropanol and high conditions of chaotropic salts, which remove water from hydrated molecules in solution. At this point, MGPs with bound nucleic acids can be magnetically separated from the residual lysed sample and unbound substances, such as polysaccharides, proteins, cell debris, and PCR inhibitors removed by subsequent washing. Purified nucleic acids may be eluted from the MGPs by applying low-salt conditions and heat. Or, other components for inactivation of virions and/or isolation of viral nucleic acid may be used as part of the system.

Also, as disclosed herein the system may comprise a station or component to detect a nucleic acid sequence specific to the non-variola Orthopoxvirus by PCR 208. In certain embodiments, qPCR is used as disclosed for the methods herein. Thus, as disclosed herein, the system may comprise primer sets and appropriate negative and positive control templates. In certain embodiments, each extracted DNA sample is analyzed with each primer/probe set separately. Or qPCR may be performed using a multiplex PCR reaction allowing for amplification of the non-variola Orthopoxvirus (e.g., VAC primers and probes) as well as positive controls (e.g., RP primers and probes and/or vaccinia primers and probes). For multiplex qPCR different fluorophores may be used to detect different amplification products. In certain embodiments, primers and probes approved by and available from the Center for Disease Control (CDC) are used. Thus, in certain embodiments, the system may comprise a Non-variola Orthopoxvirus Real-Time PCR Primer and Probe Set (VAC1) primers and probes (CDC KT0035A) and/or a RNase P Real-Time PCR Primer and Probe Set (RP) CDC KT0068. Also in certain embodiments, the system may comprise a positive control or controls. For example, in certain embodiments, the positive control may comprise a Vaccinia virus (e.g., Vaccinia (MVA)) (VAC-1 positive control) from ATCC (VR-1508). Alternatively, the system may comprise a BEI or ATCC extracted genomic (e.g., Vaccinia (MVA) Genomic, ATCC; catalog #VR-1508D) control. Also in certain embodiments, the system may comprise a RNase P positive control (i.e., PCR amplification of the RNase-P gene) to ensure adequate DNA yield with specimen collection or the DNA extraction process. For example, in certain embodiments, the system may comprise a RNase P Real-Time PCR Primer and Probe Set (RP) CDC KT0068 as a negative control. Also, in certain embodiments, the system may comprise an extraction control (EC).

For example, in certain embodiments, the non-variola orthopoxvirus forward primer may be 5′-TCAACTGAAAAGGCCATCTATGA-3′ (SEQ. ID NO. 1), the reverse primer may be 5′-GAGTATAGAGCACTATTTCTAAATCCCA-3′ (SEQ. ID NO. 2), and the probe sequence may be 5′-FAM-CCATGCAATA(T-BHQ1)ACGTACAAGATAGTAGCCAAC-Phos-3′ (SEQ ID NO. 3). The human DNA RNase P control forward primer may be 5′-AGATTTGGACCTGCGAGCG-3′ (SEQ. ID NO. 4), the RNase P reverse primer may be 5′-GAGCGGCTGTCTCCACAAGT-3′ (SEQ. ID NO. 5), and the RNase P probe may be 5′-FAM-TCCTGACCTGAAGGCTCTGCGCG-BHQ1-3′ (SEQ. ID NO. 6). qPCR may be performed using optimized cycling procedures depending on the primers and probes used. For example, in one embodiment, the PCR may comprise an initial denaturation step at 95° C. for 20 seconds (i.e., 1 cycle) followed by 40 cycles of 95° C. for 3 seconds and 63° C. for 30 seconds. See e.g., Jun. 2, 2022: Lab Advisory: CDC Publishes Non-variola Orthopoxvirus PCR Testing Procedure. In another embodiment, the PCR may comprise an initial denaturation step at 95° C. (i.e., 1 cycle) followed by 40 cycles of 95° C. for 5 seconds and 63° C. for 30 seconds. Or other probes and or primers may be used.

In certain embodiments, any of the components or stations of the system may be controlled by a computer 300. Thus, the system may be configured to perform the process of extracting and/or purifying a nucleic acid sequence in an automated manner. In one embodiment, the system configured to perform any automated process may utilize a barcode process in which reagents and/or containers are labeled appropriately with a manufacturer's barcode. The barcode may provide the system with a detailed description of the reagent and/or container that the system is utilizing. A barcode system may aid in the automated process by further providing the system with instructions on utilizing the reagent and/or container.

Or the computer 300 may control at least some of the amplification via PCR 208. In one embodiment, the system may comprise a component or station configured to perform PCR in an automated manner using the extracted and purified sample. In one embodiment, the system may be configured to receive a volume of a sample to begin PCR. The system may be further embodied in a manner that utilizes containers such as multi-well plates for PCR analysis of the sequence of interest. Reagents included as part of the system may include PCR-grade water, magnesium chloride, a fluorescent-based probe, and a forward and reverse primer set specific to the nucleic acid sequence of interest.

The system may also comprise a station or component for analysis of the results and/or to generate a report that may be shared with the subject or the subject's physician 210. In an embodiment, a computer 300 may control as least one aspect of this station. In one embodiment, the system is configured such that a defined CT is used as a method of determining that a sample contains the E9L gene. The system may calculate a CT by quantifying the number of times that a sequence of interest is amplified.

In one embodiment, the system may be configured to quantify the PCR CT cycles using fluorescence. A fluorescent probe with complementary base-pairs to the sequence of interest may anneal to the single-stranded sequence during one of the three PCR steps. A fluorescent probe may be bound to a set of complementary base-pairs. The fluorogenic probe comprising an oligonucleotide sequence may contain a reporter (e.g., a fluorophore) bound to a 5′ end and a quencher dye attached at a different position in the oligonucleotide (e.g., at or near the 3′ end or elsewhere). In one embodiment, the fluorogenic probe may anneal to a target sequence between the forward and reverse primer set. The nuclease activity of the Taq polymerase may degrade the probe, causing the reporter to separate from the quencher dye, resulting in fluorescence emission. In an embodiment, one cycle of PCR will yield one unit of fluorescence from the probe. Fluorescence, therefore, may correlate with a presence of a sequence of interest. As disclosed herein, a variety of fluorophores and/or quencher dyes may be used.

In an embodiment, a number of CTs may define whether the nucleic acid sequence of the virus is present as determined by fluorescence intensity. A CT value may be defined as a number relative to a control CT for evidence for the sequence of interest. Various embodiments of the system may determine CT units differently. In one embodiment, a CT threshold for the target primer and probe set (VAC1) may be 500,000 units while the human RNase P gene control (RP) may be 400,000 units.

Also disclosed is a computer-program product tangibly embodied in a non-transitory machine-readable storage medium including instructions configured to cause one or more data processors to run any of the components of a system or to perform any of the steps of the disclosed methods. Thus, in an embodiment, disclosed a computer-program product tangibly embodied in a non-transitory machine-readable storage medium including instructions configured to run a system comprising any one of: (a) a component for isolation of a non-variola Orthopoxvirus from a sample from a subject, the sample optionally comprising a solid substrate, and optionally using a multi-well procedure; (b) a component for purification of a nucleic acid specific to the non-variola Orthopoxvirus; (c) a component for PCR amplification of the nucleic acid sequence specific to the non-variola Orthopoxvirus; and (d) a component to output results indicating a presence or absence of the nucleic acid sequence specific to the non-variola Orthopoxvirus.

Additionally and/or alternatively, disclosed is a computer-program product tangibly embodied in a non-transitory machine-readable storage medium including instructions configured to detect a presence of a non-variola Orthopoxvirus in a subject comprising any one of the steps of: (a) obtaining a sample from the subject; and (b) detecting a nucleic acid sequence specific to the non-variola Orthopoxvirus and optionally (c) detecting a second nucleic acid sequence that serves as a control by any of the methods disclosed herein.

The systems and computer products may perform any of the methods disclosed herein. One or more embodiments described herein can be implemented using programmatic modules, engines, or components. A programmatic module, engine, or component can include a program, a sub-routine, a portion of a program, a software component, or a hardware component capable of performing one or more stated tasks or functions. As used herein, a module or component can exist on a hardware component independently of other modules or components. Alternatively, a module or component can be a shared element or process of other modules, programs, or machines.

As will be appreciated by one of skill in the art, the present disclosure may be embodied as an apparatus, a method, data or signal processing system, or computer program product. Accordingly, the present disclosure may take the form of an entirely software embodiment, or an embodiment combining software and hardware aspects. Furthermore, certain embodiments of the present disclosure may take the form of a computer program product on a computer-usable storage medium having computer-usable program code means embodied in the medium. Any suitable computer readable medium may be utilized including hard disks, CD-ROMs, optical storage devices, or magnetic storage devices.

The computer-usable or computer-readable medium may be, but is not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). The computer-usable or computer-readable medium could even be paper or another suitable medium, upon which the program is printed, as the program can be electronically captured, via, for instance, optical scanning of the paper or other medium, then compiled, interpreted, or otherwise processed in a suitable manner if necessary, and then stored in a computer memory.

Computer program code for carrying out operations of the present disclosure may be written in an object-oriented programming language such as Java7, Smalltalk, Python, Labview, C++, or VisualBasic. However, the computer program code for carrying out operations of the present disclosure may also be written in conventional procedural programming languages, such as the “C” programming language or even assembly language. The program code may execute entirely on the user's computer, partly on the user's computer, as a stand-alone software package, partly on the user's computer and partly on a remote computer or entirely on the remote computer. In the latter scenario, the remote computer may be connected to the user's computer through a local area network (LAN) or a wide area network (WAN), or the connection may be made to an external computer (for example, through the Internet using an Internet Service Provider).

The disclosed systems and/or computer software allow for high throughput screening for a non-variola Orthopoxvirus (e.g., a monkeypox virus). Using the disclosed multiwell extraction process for virion isolation, followed by multiwell purification of viral nucleic acid and/or multiplex PCR allows for assaying multiple samples individually. For example, in certain embodiments, 93 individual samples may be processed in a single 96-well plate. This can allow for a single laboratory to process hundreds (e.g., 4 or more plates) per day. For example, in certain embodiments, the methods allow for processing at least 20, or 40, or 60, or 80, or 100, or 150, or 200, or 250, or 300, or 350, or 400, or 450, or 500 or more samples in one day. Or, in some cases duplicates or triplicates of a sample may be evaluated.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1—Assay Controls and Preparation

Non-variola Orthopoxvirus Real-Time PCR primers and probes as well as RNase P Real Time PCR primers and probes obtained from CDC (CDC KT00035A; KT0068) were used. Additional reagents included Human Genomic DNA from human blood (buffy coat) (Roche Applied Science, Penzberg, Germany; Cat. No. 11691112001); Vaccinia virus (e.g., Vaccinia (MVA) Genomic, ATCC; Catalog #VR-1508D).

### Example 2—Non-variola Orthopoxvirus Extraction Using MagNa Pure 96

The MagNA Pure (Roche Diagnostics, Indianapolis, IN) allows for the automated isolation of nucleic acids from crude sample material using specifically designed reagent kits. Key steps of MagNA Pure 96 NA isolation are as follows:
1. Sample material is lysed, nucleic acids are released, and nucleases are denatured.
2. Nucleic acids bind to the silica surface of the magnetic glass particles (MGPs) in the presence of isopropanol and high conditions of chaotropic salts, which remove water from hydrated molecules in solution.
3. MGPs with bound nucleic acids are magnetically separated from the residual lysed sample.
4. Unbound substances, such as polysaccharides, proteins, cell debris, and PCR inhibitors are removed by subsequent washing.
5. Purified nucleic acids are eluted from the MGPs by applying low-salt conditions and heat.

### Specimen Requirements

Dry swabs from a patient suspected of being infected with monkeypox, preferably two dry swab samples. Alternatively, one swab may be submitted in liquid viral media (UTM/VTM). If dry swabs are submitted, only one dry swab is used for testing according to this procedure. The second swab may be referred to CDC or other public health laboratories for additional testing, if required. Store residual specimens at 2-8° C. or ≤−20° C. If a swab is submitted in VTM/UTM, 100 μL of transport media is extracted.

Acceptable swab types include synthetic tip swabs with aluminum or plastic shafts shipped with or without media.

### Collection Instructions

Specimens were collected using the swab within the collection kit by vigorously swabbing the skin for 1-2 minutes. Collection was repeated with a second swab over the same lesion. Swabs were then placed immediately into the collection tube, refrigerated at 2-8° C. and shipped overnight to the laboratory.

### Specimen Storage

Specimens can be stored up to 7 days refrigerated (2-8° C.) or frozen (−20° C. or lower) prior to testing. Extracted nucleic acids should be stored at −70° C.

### Swab Elution and DNA Extraction Protocol for Samples Submitted by Dry Swabs Using the ProMega SlicPrep Protocol

Using a multichannel pipet, 300 μL of PBS was added to the SlicPrep plate prior to adding cut swabs, leaving some wells reserved for assay controls.

Using sterile tweezers, the dry swabs were carefully removed from the submitted container and placed in a clean 2 mL ampule secured in a specimen rack. The swab shafts were then cut for each sample close to the level of elution tray. The dry swabs were then removed from the 2 mL ampule and placed into a desired position of the Promega SlicPrep plate (Promega, Madison, WI). All swabs were positioned to be fully seated in the top section of the SlicPrep plate. Alternatively, swabs may be positioned by sealing the plate and centrifuging. Swabs were allowed to elute into the PBS for at least 10 minutes. After inserting the white elution tray collar, the 96-well tray was covered with sealing film and centrifuged at 1900×g for 1 minute to ensure sample collection at the bottom of each well. Centrifugation was conducted using sealed/capped cups which were opened only in the Biosafety cabinet.

At this point, the upper plastic container with swabs and white elution tray collar was discarded and 100 μL of MagNA Pure LC Total Nucleic Isolation Kit Lysis/Binding Buffer added to the MagNA Pure 96 source. Next, 100 μL of the swab elution was removed and added to the MagNA Pure 96 source plate and slowly mix.

For addition of negative extraction controls, the swab elution/MagNA Pure Lysis Binding Buffer was allowed to incubate for 15 minutes at room temperature to deactivate monkeypox virus and the plate centrifuged at 550×g for <30 seconds to collect all the liquid at the bottom of the wells.

### Swab Elution and DNA Extraction Protocol for Samples Submitted by Dry Swabs Using the SETS Tube Protocol

Using sterile scissors, swabs were cut near the top of the swab material and placed into a labeled microcentrifuge tube. Following, the swab was hydrated with 300 μL of sterile PBS for 5-10 minutes. Using sterile tweezers, the swab was transferred to the inner SETS tube placed over a microcentrifuge tube. Any remaining PBS was transferred to the SETS tube with an aerosol-barrier micropipette tube. The SETS tube containing the swab was centrifuged using a microcentrifuge (1900×g) for 1 minute to collect the eluate. The above steps were repeated with additional swabs requiring elution.

To a MagNA Pure Source plate, 100 μL of MagNA Pure Lysis/Binding Buffer was added to each well. Next, 100 μL of elution from each SETS tube was removed and added to the MagNA Pure Source plate and mixed slowly. The swab elution/MagNA Pure LC Lysis/Binding Buffer was allowed to incubate for 15 minutes at room temperature for virus deactivation. Then, the plate was briefly spun in a plate centrifuge (preferably at 500×g for <30 seconds) to collect all of the liquid in the bottom of the wells.

### Swab Elution and DNA Extraction Protocol for Samples Submitted in Liquid Viral Media

Using a multichannel pipette, 100 μL of MagNA Pure LC Lysis/Binding buffer was aliquoted to a pre-selected set of wells of a clean MagNa Pure plate source plate and set aside. Briefly, the swab was vortexed in UTM/VTM and quick spun to collect all the viral media in the bottom of the tube. Working one specimen at a time, 100 μL of the viral media was aliquoted to the appropriate well in the MP96 source plate containing 100 μL of Lysis-Binding buffer. Slowly the specimen and lysis/binding buffer were mixed using, for example, a pipet. The plate was incubated for 15 minutes at room temperature for virus inactivation and then, the plate was sealed using, for example, sealing film, and the plate was briefly spun in a plate centrifuge (about 500×g for less than or about 30 seconds) to collect all of the liquid in the bottom of the wells.

### Example 3—Real-Time PCR

Using a QuantStudio 7 Flex Real-Time PCR System (Thermo Fisher Scientific, Waltham, MA), PCR for an extracted and purified sequence was performed essentially as follows. In addition to E9L, patient samples were extracted and amplified for RNase P, which serves as a sample-specific internal control (IC). In order for the patient specimen to generate an IC result of ‘Pass’, the IC must have a Ct value less than 40, or the VAC1 result is positive. If the VAC1 is positive the status of the IC is not employed. An assay run is valid if all controls are deemed valid. If controls are not valid, re-extraction and/or re-amplification of a batch was attempted. If the specimen generated an IC result >40, poor collection or PCR inhibition was suspected. In this case the samples were routed for re-extraction. If the repeat extraction produced a failing IC (>40) and negative VAC1, the result was reported as “INCONC.” If the repeat extraction produced a passing IC (<40), the sample was reported as appropriate. An assay run was considered valid if all controls are valid.

All samples that were negative for the target analyte (VAC1) had a passing IC result. If the analysis report generated an INVALID result on either Negative Extraction Control (NEC) for the following scenarios:
1) If VAC1: <40 then RP was not considered and all specimens that produced a negative target (VAC1) result and an acceptable RP result as were reported as Not Detected and extraction repeated for all positive specimens.
2) If VAC1: >40 or Undetermined and RP>40 then reported all specimens that produced a positive target result and an acceptable RP result as Detected; reported all specimens that produced a negative target result and an acceptable RP result as Not Detected; and repeated all specimens that gave an RP>40 from extraction.
3) If the analysis report generates an INVALID result on No Template Control (NTC) due to VAC1: <40: then reported all specimens that produced a Negative target result and an acceptable RP result as Not Detected and (b) repeat from extraction all positive specimens.
4) If the analysis report generates an INVALID for the Positive Amplification Control (PAC), repeat all specimens from amplification and (a) report all specimens that produced a Positive target result and an acceptable RP result as Detected; (b) repeat from amplification all specimens that produced a negative target result; and (c) if a PAC control failure and no positive specimens, pull all specimens for repeat extraction.

Repeat testing can also be performed as a quality control check for specimens with a higher-than-expected VAC1 Ct value to ensure reproducibility of the result and to avoid false positives. Repeat testing is performed for specimens that initially show an Equivocal or Inconclusive result.

Specimens with VAC1 Ct values >30.0 but less than 37.0 may be subject to repeat, especially if they are found within a cluster of other positive samples.

Specimens with a repeat VAC1 result of ≤37.0 are reported as Detected. In these cases, RP is not required to be ≤40.

Specimens with a repeat VAC1 result of >37.0 to ≤40.0 are reported as Equivocal. In these cases, RP is not required to be ≤40.

Specimens with a repeat VAC1 result of ≥40.0 and an RP≤40.0 are reported as Not Detected.

Table 1 displays results according to a provided CT from VAC1 and RP.

TABLE 1
CT values and corresponding reporting according to the range of CT values.

| | VAC1 CT | RP CT | REPORT |
|---|---|---|---|
| Assay | ≤37.0 | ≤40 | Positive |
| Interpretation | >40 or none | ≤40 | Negative |
| | >37.0-≤40.0 | >40 or none | Equivocal |
| | None | None | Inconclusive |

### Example 4—at Home Collection of Samples

This example describes the development of a kit for self-collection of samples. The Monkeypox PCR Test Home Collection Kit is intended for use by individuals presenting with acute, generalized pustular or vesicular rash suspected of Monkeypox illness for self-collection of lesion swab specimens in media at home. The swab specimen is placed in media and transported to the laboratory for testing non-variola Orthopoxvirus DNA extracted from the specimens.

The kit is intended for use by individuals 18 years of age and older (self-collected). Self-collected specimens are transported at ambient temperature in transport media for testing at a central laboratory. Non-variola Orthopoxvirus DNA from the lesion swab is maintained in the specimen packaging and is suitable for testing using the methods and systems disclosed herein.

The kit is used to collect non-variola Orthopoxvirus DNA from lesion swab samples; it can also be used for transportation and short-term room temperature storage of a sample. The kit is a non-invasive alternative for collection of a high quality and quantity of non-variola Orthopoxvirus DNA from individuals who are suspected of infection for use for testing using the methods and systems disclosed herein.

The kit is composed of a shipping box, pre-labeled return envelope, collection and shipping directions, specimen collection materials (swab and transport tube), a specimen biohazard bag containing an absorbent sheet, and a specimen confirmation form. Instructions are included in the kit to direct the home users on how to appropriately collect the lesion swab specimen and place it in the transport tube, how to properly package the specimen and how to mail the specimen back to the laboratory using the pre-labeled FedEx return envelope.

The kit can be requested by a physician and sent directly to the intended user. The individual using the kit to collect a lesion swab sample performs the following steps as stated in the enclosed instructions to collect the sample:
1) The user is instructed to wash and dry their hands.
2) The user is instructed to take the swab out of its package.
3) The user is instructed to not touch the tip of the swab or touch any other surface with the swab tip. If the swab touches any other surface, the user is instructed to request a new kit. The user is instructed to not clean the lesion before collecting the sample.
4) The user is instructed to slowly swirl the swab while pressing firmly across the entire lesion at least 3 times making sure to press firmly with the swab to collect the lesion material. If there are multiple lesions, they are instructed to swab one at an intermediate stage (not brand new, not old and healing).
5) The user is instructed to unscrew the top of the collection tube and hold the swab in one hand and the collection tube in the other, being careful not to spill the liquid. If the liquid spills, they are instructed to request a new kit. The user is instructed to not drink the liquid.
6) The user is instructed to insert the swab into the collection tube. The end of the swab that was used to swab the lesions should be placed into the tube first. The user is instructed to break the swab at the score line to allow the tube to be tightly closed and to screw the top of the collection tube back on tightly.
7) The user is instructed to wash and dry their hands thoroughly again and insert the collection tube into the biohazard specimen bag and then to close using the zip lock seal. The user is instructed to not remove the absorbent sheet from the biohazard specimen bag.
8) The user is instructed to fill out the specimen confirmation form in ink, and to include the date and time of collection in the Patient Section.
9) The user is instructed to place the specimen bag and specimen confirmation form into the white shipping box and close the lid. The user is instructed to place the shipping box into a return pack (e.g., FedEx or other carrier) and to remove the adhesive cover strip and seal the FedEx return pack closed. The user is instructed to not place the collection tube directly in the return pack for shipment.
10) The user is instructed to deliver the postage paid, pre-addressed return pack to a pre-identified drop box. The user is instructed that it is important to bring the sample to a drop box on the same day it is collected before the last pickup Monday-Thursday (no weekends). As an option to using a drop box, a flyer is provided in the kit instructing the user how to schedule a pickup on the day of collection.

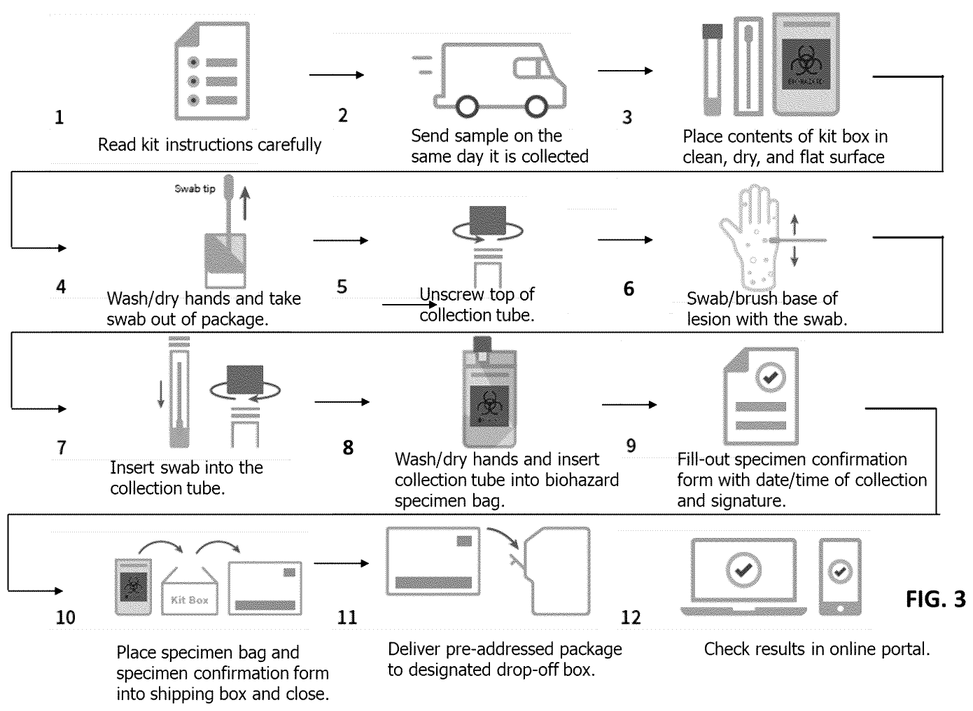

An example of user instructions summary is shown in FIG. 3

Prior to acceptance for testing by the laboratory (e.g., using the Non-variola Orthopoxvirus Real-time PCR Assay disclosed herein, samples received at the clinical laboratory are subjected to the following accessioning procedure:
1) The return package is opened, and the kit box is removed. The kit box is opened and the biohazard specimen bag containing the sample is removed.
2) Any potential issues with the specimen that would prevent it from being accessioned are identified. Issues include but are not limited to: (a) No swab included with the collection tube; and (b) Collection tube leaked, resulting in no sample for testing.

### Sample Collection Control

As described herein, the Non-variola Orthopoxvirus Real-time PCR Assay utilizes the RNase P gene in patient samples as an internal control that also serves as a sample collection control for the Monkeypox PCR Test Home Collection Kit.

### Test Result Reporting

All test results may be reported to healthcare providers and relevant public health authorities in accordance with local, state, and federal requirements. Core diagnostic data elements may be collected for all tests, which have been defined by the Department of Health and Human Services (HHS), along with technical specifications for implementation for lab-based and non-lab-based tests.

### Performance Evaluation

The shipping stability of lesion swab samples when using the disclosed Monkeypox PCR Test Home Collection Kit was demonstrated by performing a winter and summer temperature excursion stability study using positive and negative contrived specimens as outlined in FDA recommendations for home collection devices.

### Preparation of Test Specimens

Negative Contrived Samples—Residual, clinician-collected lesion swabs in UTM that were tested by the NVO assay and reported as Negative for Orthopoxvirus DNA were used to create negative UTM pools. Specimens selected had RP Ct values below 24.00 and when combined to a pool, were re-screened to confirm the Orthopoxvirus DNA Negative result and to obtain RP baseline Ct. This negative pool was used for both negative contrived specimen preparation and positive contrived preparation.

Internal control (RP) was introduced into contrived negative samples by spiking each of the 10 blank UTM replicates with 101 μL of negative UTM pool.

Positive Contrived Samples—Twenty contrived low positive samples (2×LoD) and 10 contrived high positive samples (10×LoD) were created by diluting Vaccinia virus (ATCC-1566) into the negative pooled matrix generated as described above. Vaccinia virus (ATCC-1566) was quantified at a starting concentration of 3.67e10⁵ cp/μL. A working stock was prepared by diluting 5 μL into 1995 μL UTM, to achieve a concentration of 917.5 cp/μL. 2×LoD dilution: 20 μL working stock pipetted into 3000 μL blank UTM tubes. 10×LoD dilution: 101 μL working stock pipetted into 3000 μL blank UTM tubes. Winter and Summer Shipping profiles are shown in Tables 2 and 3 below.

TABLE 1
Winter Shipping Profile Summary

| Temperature | Cycle Period | Cycle Period Hours | Total Time Hours |
|---|---|---|---|
| −10° C. | 1 | 8 | 8 |
| 18° C. | 2 | 4 | 12 |
| −10° C. | 3 | 2 | 14 |
| 10° C. | 4 | 36 | 50 |
| −10° C. | 5 | 6 | 56 |
| 10° C. | 6 | 16 | 72 |
| 22° C. | 7 | 72 | 144 |

TABLE 2
Summer Shipping Profile Summary

| Temperature | Cycle Period | Cycle Period Hours | Total Time Hours |
|---|---|---|---|
| 40° C. | 1 | 8 | 8 |
| 22° C. | 2 | 4 | 12 |
| 40° C. | 3 | 2 | 14 |
| 35° C. | 4 | 36 | 50 |
| 40° C. | 5 | 6 | 56 |
| 35° C. | 6 | 16 | 72 |
| 22° C. | 7 | 72 | 144 |

### Acceptance Criteria and Results

Negative Contrived Samples: 100% agreement with expected results. Positive Contrived Samples: Low Positive Samples: ≥:95% agreement with expected results. High Positive Samples: 100% agreement with expected results. It was found that all negative samples were 10000 concordant after both the winter and summer shipping excursions. All positive samples were 100% concordant between the 0 hr and 144 hr tests. Results of the positive samples are summarized in Table 3 and Table 4.

TABLE 3
Winter Shipping Excursion Results Summary

| Contrived | T-0 hr | | T = 144 hr | | |
|---|---|---|---|---|---|
| Samples | Pos/Total | Mean Ct | Pos/Total | Mean Ct | Delta Ct |
| 2X LoD | 20/20 | 34.51 | 20/20 | 35.60 | 1.09 |
| | (100%) | | (100%) | | |
| 10X LoD | 10/10 | 31.36 | 10/10 | 33.06 | 1.70 |
| | (100%) | | (100%) | | |

TABLE 4
Summer Shipping Excursion Results Summary

| Contrived | T-0 hr | | T = 144 hr | | |
|---|---|---|---|---|---|
| Samples | Pos/Total | Mean Ct | Pos/Total | Mean Ct | Delta Ct |
| 2X LoD | 20/20 | 33.96 | 19/20 | 35.82 | 1.86 |
| | (100%) | | (100%) | | |
| 10X LoD | 10/10 | 31.04 | 10/10 | 32.87 | 1.83 |
| | (100%) | | (100%) | | |

The data from the winter and summer excursion study supports the stability of lesion swab samples collected with the kit for up to 144 hours in an ambient temperature shipping environment during winter and summer followed by 72 hours at room temperature after laboratory receipt. Instructions for the use of the kit are shown in FIG. 3.

### Example 5—Kit Verification

A specific study to compare the results generated from the intended users self-collected swabs to healthcare provider (HCP) collected swabs (same lesion for each subject) and to demonstrate intended users can successfully self-collect lesion swabs in a simulated home setting using the applicable components of the kit may be performed as follows.

The study can be conducted at one or more US clinical sites in eligible subjects presenting to the clinic with signs and symptoms consistent with Mpox. After eligibility is confirmed, written informed consent can be obtained and the study procedures explained to the participant by the study staff. Subjects may provide basic demographics and symptoms in support of their sample collection. Enrolled participants are provided with a Mpox PCR Home Collection Test Kit which contain the following:

Mpox PCR Home Collection Test Kit Contents:
Specimen Confirmation Form (SCF)
1 collection swab
1 swab transport media tube
1 biohazard specimen bag in which the sample is sealed for purposes of safe transport after the sample is collected
1 pre-labeled box for purposes of transporting the sample from the home to the lab
1 pre-paid shipping envelope
The instructions for use (IFU) including a description of the collection kit contents, instructions for performing the self-collection, instructions for resealing the collection device for transport and return shipping instructions.

Subjects collect their sample in a simulated home environment with a drop box to simulate shipping their self-collected sample. Upon receipt of the collection kit, the participant confirms the contents of the kit and the information on the SCF. After confirming the contents and information, the subject follows the collection instructions on the kit's IFU to self-collect one swab from one lesion. Subjects then package the sample for shipping.

Study staff observe participants as they perform and describe their understanding of the tasks associated with sample collection, packaging, and shipping and will document difficulties and use errors. Each participant completes a questionnaire evaluating the entire workflow. The kits containing the subject self-collected samples are shipped by the study site staff on the date of collection to the designated laboratory.

To minimize the number of required lesion swab collections, if a standard-of-care test is ordered during the same study visit, the standard-of-care (SoC) results may be used as the comparator results for the analysis.

After self-collection is completed, an HCP collects one lesion swab following their current (or SoC) collection protocol from the same lesion that was swabbed by the subject. The HCP-collected samples are shipped by the study site laboratory facility currently conducting the disclosed Mpox (Orthopoxvirus), deoxyribonucleic acid (DNA), polymerase chain reaction (PCR) Assay on the date of collection.

The self-collected sample kits can be assessed upon receipt at the laboratory for compliance with the packaging and shipping instructions and any deviations will be noted. Both the self-collected and the HCP-collected samples are tested using the Mpox Assay disclosed herein with the results generated using the HCP-collected sample considered the true result. If the HCP-collected sample is also considered the SoC sample for a subject, the results of the HCP-collected sample can be reported back to the treating physician as is currently done for all commercial Mpox Assay samples. All collected study data are de-identified. Endpoints

Primary Endpoint: To evaluate the concordance between the results generated by the disclosed Mpox Assay analysis of a self-collected swab sample and an HCP-collected swab sample from skin lesions of patients suspected to be infected with Mpox.

Secondary Endpoint: To evaluate the number of self-collected samples received by the designated laboratory that meet all the acceptability criteria for testing using the disclosed Mpox Assay.

Tertiary Endpoint: To summarize the questionnaire results and to ensure the Mpox PCR Home Collection Test Kit meets acceptability criteria or to modify the sample collection instructions as needed.

### Study Population, Clinical Site, Sample Size and Inclusion Exclusion Criteria

The study population may comprise male, female or transgender patients ≥18 years of age who present to the participating study clinic with signs and symptoms of Mpox viral infection and who meet all study inclusion criteria and no exclusion criteria will be considered for enrollment. Subjects participating in other clinical trials are not necessarily excluded provided that those trials do not involve prior swab collection of lesion samples prior to this study. Subjects with prior medical training may be excluded. Subjects with known Mpox diagnostic test results prior to the study visit will not be excluded. Written consent and enrollment are performed using standard procedures. Subjects may be enrolled until a minimum of 30 positive and 30 negative subjects (assessing only one study lesion per subject) as determined by the HCP-collected specimen results have been acquired. The total sample size maybe driven by the prevalence of infection within the communities where the study is being conducted.

Inclusion and Exclusion Criteria may be as follows:

### Inclusion Criteria:

Subject is male or female or transgender and ≥18 years of age.
Subject agrees to provide written informed consent.
Subject agrees to provide the required data in support of their participation.
Subject can read and understand English.
Subject presents to the clinic with signs and symptoms consistent with Mpox infection.
Subject has at least one lesion.

### Exclusion Criteria:

Previous participation in this protocol.
Subject has prior medical (e.g., a nurse or physician) training.
Subject is not able or willing to contribute the required swab samples for testing.

Where the Mpox Kit is labeled for prescription use only, self-collection at home will occur following a clinician's order. Once the physician order is placed, the home collection kit can be mailed to the patient, who performs the sample collection and mail it back to the designated laboratory. Test results are relayed back to the ordering physician.

All positive, inconclusive and equivocal test results will be communicated to the individual through a healthcare professional (either the ordering physician or the selected results communication vendor) and are then delivered to the user via their website account.

### Study Procedures

After written informed consent is obtained, the required demographic and symptom data documented, the Mpox Kit is provided to the subject. Each kit can be labeled with a unique identification barcoded number and will be externally labelled as an investigational device as provided by federal law. In certain cases as authorized by the site PI, a delegated staff member will observe participants as they perform the tasks associated with sample collection, sample packaging and shipment and will document difficulties and use-errors. Following the self-collection, the HCP will collect a lesion swab.

Upon receipt of the specimen kit, patients that ordered the kit online can use the internet to register their kit. Patients receiving their kit ordered by their treating physician can confirm that their personal information listed on the accompanying SCF and specimen tube label is accurate but will not need to register their kits. Each kit can be externally labelled as an Investigational Device as required by law.

Each subject provides 2 lesion swab samples from the same lesion to be analyzed for the study. To minimize the number of required lesion swab collections, if SoC is ordered for the subject during the same study visit, results derived from HCP-collected sample can be used for both SoC and the comparator.

After confirming all contents of the kit are present, the participant follows the collection instructions. Subjects are assessed as to their understanding of how the swab is to be collected via a comprehension questionnaire.

Study staff may observe participants as they perform the tasks associated with SCF review/completion, sample collection, packaging and shipping and will document difficulties and use-errors. Each participant can complete a comprehension questionnaire evaluating each aspect of the sample collection process from SCF review/completion (adding the date and time of sample collection where indicated) to their understanding of shipping the collected sample. The kits containing the self-collected samples will be shipped by the study staff on the date of collection on behalf of the subject to the designated laboratory for accessioning prior to analysis.

For HCP-collected lesion swabs, the collector follows the comparator test IFU and ships the sample through the normal commercial channels for samples to be tested for Mpox. Each HCP collected lesion swab sample will be obtained from the lesion as used for each self-collected lesion swab sample. If SoC is ordered for the subject, the results of the HCP-collected sample will be reported back to the treating physician.

Performance is assessed based on the comparison of same subject self-collected lesion swab and HCP-collected lesion swab results as analyzed using the disclosed systems and methods and on tabulation of study subject, observer and accessioning responses. It is expected that a majority (>50%) of the participants can successfully follow the collection instructions, that more than 85% of the specimens are received by the laboratory in an acceptable condition for testing, and of those received and approved for testing, the concordance between self-collected and HCP-collected samples will be ≥95% PPA and ≥95% NPA. The sample accessioning acceptability criteria being studied are driven by the design of the home collection kits and IFU that take into account the previously conducted temperature excursion studies on sample stability for analysis of a skin lesion swab with the disclosed systems and methods. The number and proportion of samples received and considered as valid and invalid for analysis will be determined. Additionally, any difficulties observed or experienced will be used to consider labeling and changes to the specimen collection instructions.

All documents referred to in this specification are herein incorporated by reference. Various modifications and variations to the described embodiments of the disclosure will be apparent to those skilled in the art without departing from the scope of the invention. Although the present disclosure has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention. Where a conflict exists between the instant application and a reference provided herein, the instant application shall dominate.

### Example 6—Exemplary Embodiments

Exemplary embodiments provided in accordance with the presently disclosed subject matter include, but are not limited to, the claims and the following embodiments.

Embodiment 1: A method for detecting a presence of a non-variola Orthopoxvirus in a subject comprising: (a) obtaining a sample from the subject; (b) detecting a first nucleic acid sequence specific to the non-variola Orthopoxvirus; and (c) detecting a second nucleic acid sequence that serves as a control.

Embodiment 2: The method of Embodiment 1, wherein the non-variola Orthopoxvirus is monkeypox (Mpox).

Embodiment 3: The method of Embodiment 2, wherein the first nucleic acid sequence comprises sequences specific to the E9L gene of the non-variola Orthopoxvirus.

Embodiment 4: The method of Embodiment 1, wherein detecting the first nucleic acid sequence comprises the step of real-time PCR.

Embodiment 5: The method of Embodiment 2, wherein the second nucleic acid comprises sequences specific to the human RNase P gene and/or a vaccinia virus.

Embodiment 6: The method of Embodiment 1, wherein detecting the first nucleic acid sequence comprises isolation of the non-variola Orthopoxvirus from a solid substrate.

Embodiment 7: The method of Embodiment 6, wherein isolation of the non-variola Orthopoxvirus from the solid substrate comprises a multi-well extraction procedure.

Embodiment 8: The method of Embodiment 6, wherein the solid substrate comprises a dry swab.

Embodiment 9: The method of Embodiment 8, wherein the dry swab is a synthetic tip swab with an aluminum shaft or a synthetic tip swab with a plastic shaft.

Embodiment 10: The method of Embodiment 1, wherein the step of obtaining the sample comprises self-collection of the sample by the subject.

Embodiment 11: The method of Embodiment 1, further comprising purification of the first nucleic acid sequence by binding the nucleic acid to silica beads.

Embodiment 12: The method of Embodiment 1, wherein multiple individual samples are processed simultaneously.

Embodiment 13: The method of Embodiment 12, wherein at least 2, or 5, or 10, or 15, or 20, or 25, or 30 or 35, or 40, or 45, or 50, or 55, or 60, or 65, or 70, or 75 or 80, or 85, or 90 or more samples are processed simultaneously.

Embodiment 14: The method of Embodiment 5, further comprising multiplex qPCR for at least two of the E9L gene, the human RNase P gene or the vaccinia virus sequences.

Embodiment 15: The method of Embodiment 1, wherein detecting the first nucleic acid sequence comprises isolation of the non-variola Orthopoxvirus from a sample submitted in liquid viral media.

Embodiment 16: The method of Embodiment 15, wherein isolation of the non-variola Orthopoxvirus from the solid substrate comprises a multi-well extraction procedure.

Embodiment 17: A system for detecting a presence of a non-variola Orthopoxvirus in a sample from a subject comprising: (a) a component for isolation of the non-variola Orthopoxvirus from a solid substrate comprising the sample using a multi-well procedure; (b) a component for purification of a nucleic acid sequence specific to the non-variola Orthopoxvirus; (c) a component for PCR amplification of the nucleic acid sequence specific to the non-variola Orthopoxvirus; and (d) a component to output results indicating a presence or absence of the nucleic acid sequence specific to the non-variola Orthopoxvirus.

Embodiment 18: The system of Embodiment 17, further comprising a component for self-collection of the sample.

Embodiment 19: The system of Embodiment 18, wherein the component for self-collection of the sample comprises a kit.

Embodiment 20: The system of Embodiment 19, wherein the kit comprises: a swab for collection of the sample from a subject; a transport container; a biohazard bag; optionally, external packaging for the subject to mail the sample to a laboratory for testing; and instructions for use.

Embodiment 21: The system of Embodiment 17, wherein the non-variola Orthopoxvirus is monkey pox.

Embodiment 22: The system of Embodiment 17, wherein the nucleic acid sequence specific to the non-variola Orthopoxvirus comprises sequences specific to the E9L gene of the non-variola Orthopoxvirus.

Embodiment 23: The system of Embodiment 17, further comprising detecting nucleic acid sequences specific to the human RNase P gene and/or a vaccinia virus.

Embodiment 24: The system of Embodiment 17, wherein the PCR is real-time PCR.

Embodiment 25: The system of Embodiment 17, wherein the solid substrate comprises a tissue swab.

Embodiment 26: The system of Embodiment 17, wherein the component for purification of the nucleic acid specific to the non-variola Orthopoxvirus comprises silica beads.

Embodiment 27: The system of Embodiment 17, wherein at least one of the components is controlled by a computer.

Embodiment 28: The system of Embodiment 17, wherein multiple individual samples are processed simultaneously.

Embodiment 29: The system of Embodiment 17, wherein at least 2, or 5, or 10, or 15, or 20, or 25, or 30 or 35, or 40, or 45, or 50, or 55, or 60, or 65, or 70, or 75 or 80, or 85, or 90 or more samples are processed simultaneously.

Embodiment 30: The system of Embodiment 17, further comprising a component for multiplex real time PCR for at least two of the E9L gene, the human RNase P gene or vaccinia virus sequences.

Embodiment 31: A kit for self-collection of a sample for determining the presence of a non-variola Orthopoxvirus in a subject comprising: a swab for collection of the sample from the subject; a transport container; a biohazard bag; optionally, external packaging for the subject to mail the sample to a laboratory for testing; and instructions for use.

Embodiment 32: A computer-program product tangibly embodied in a non-transitory machine-readable storage medium including instructions configured to cause one or more data processors to perform any of the steps of Embodiments 1-16 or to run any of the systems of Embodiments 17-30 or to use the kit of Embodiment 31.

Embodiment 33: A computer-readable medium comprising stored instructions, wherein the instructions when executed by a processor cause the processor to perform any of the steps of Embodiments 1-16.

Embodiment 34: A computer-readable medium comprising stored instructions, wherein the instructions are readable by the system of Embodiments 17-30.

Embodiment 35: A computer-readable medium comprising stored instructions, wherein the instructions when executed by a processor can be used by the kit of Embodiment 31.

That which is claimed is:

1. A method for detecting a presence of a non-variola Orthopoxvirus in a subject comprising:
(a) obtaining a sample from the subject;
(b) detecting a first nucleic acid sequence specific to the non-variola Orthopoxvirus; and
(c) detecting a second nucleic acid sequence that serves as a control.

2. The method of claim 1, wherein the non-variola Orthopoxvirus is monkeypox (Mpox).

3. The method of claim 2, wherein the first nucleic acid sequence comprises sequences specific to the E9L gene of the non-variola Orthopoxvirus.

4. The method of claim 1, wherein detecting the first nucleic acid sequence comprises the step of real-time PCR.

5. The method of claim 2, wherein the second nucleic acid comprises sequences specific to the human RNase P gene and/or a vaccinia virus.

6. The method of claim 1, wherein detecting the first nucleic acid sequence comprises isolation of the non-variola Orthopoxvirus from a solid substrate.

7. The method of claim 6, wherein isolation of the non-variola Orthopoxvirus from the solid substrate comprises a multi-well extraction procedure.

8. The method of claim 6, wherein the solid substrate comprises a dry swab.

9. The method of claim 8, wherein the dry swab is a synthetic tip swab with an aluminum shaft or a synthetic tip swab with a plastic shaft.

10. The method of claim 1, wherein the step of obtaining the sample comprises self-collection of the sample by the subject.

11. The method of claim 1, further comprising purification of the first nucleic acid sequence by binding the nucleic acid to silica beads.

12. The method of claim 1, wherein multiple individual samples are processed simultaneously.

13. The method of claim 12, wherein at least 20 samples are processed simultaneously.

14. The method of claim 5, further comprising multiplex qPCR for at least two of the E9L gene, the human RNase P gene or the vaccinia virus sequences.

15. The method of claim 1, wherein detecting the first nucleic acid sequence comprises isolation of the non-variola Orthopoxvirus from a sample submitted in liquid viral media.

16. The method of claim 15, wherein isolation of the non-variola Orthopoxvirus from the solid substrate comprises a multi-well extraction procedure.

17. A system for detecting a presence of a non-variola Orthopoxvirus in a sample from a subject comprising:
(a) a component for isolation of the non-variola Orthopoxvirus from a solid substrate comprising the sample using a multi-well procedure;
(b) a component for purification of a nucleic acid sequence specific to the non-variola Orthopoxvirus;
(c) a component for PCR amplification of the nucleic acid sequence specific to the non-variola Orthopoxvirus; and
(d) a component to output results indicating a presence or absence of the nucleic acid sequence specific to the non-variola Orthopoxvirus.

18. The system of claim 17, further comprising a component for self-collection of the sample.

19. The system of claim 17, wherein at least one of the components is controlled by a computer.

20. A kit for self-collection of a sample for determining the presence of a non-variola Orthopoxvirus in a subject comprising:
a swab for collection of the sample from the subject;
a transport container;
a biohazard bag;
optionally, external packaging for the subject to mail the sample to a laboratory for testing; and
instructions for use.


SEQUENCE LISTING (6 sequences)

SEQ ID NO: 1 - DNA, 23 bp, synthetic construct
  source 1..23: mol_type = other DNA
tcaactgaaaaggccatctatga

SEQ ID NO: 2 - DNA, 28 bp, synthetic construct
  source 1..28: mol_type = other DNA
gagtatagagcactatttctaaatccca

SEQ ID NO: 3 - DNA, 32 bp, synthetic construct
  source 1..32: mol_type = other DNA
ccatgcaatatacgtacaagatagtagccaac

SEQ ID NO: 4 - DNA, 19 bp, synthetic construct
  source 1..19: mol_type = other DNA
agatttggacctgcgagcg

SEQ ID NO: 5 - DNA, 20 bp, synthetic construct
  source 1..20: mol_type = other DNA
gagcggctgtctccacaagt

SEQ ID NO: 6 - DNA, 23 bp, synthetic construct
  source 1..23: mol_type = other DNA
ttctgacctgaaggctctgcgcg